(19) United States
(12) Patent Application Publication
(10) Pub. No.: US 2010/0028409 A1
(43) Pub. Date: Feb. 4, 2010

(54) Barrier Membranes For Guided Bone Regeneration

(75) Inventors: Doug-Youn Lee, Gyeonggi-do (KR); Sang-Bae Lee, Gyeonggi-do (KR); Kyeong-Jun Park, Gyeonggi-do (KR); Jeong-Jong Park, Gyeonggi-do (KR)

Correspondence Address: THE NATH LAW GROUP, 112 South West Street, Alexandria, VA, 22314, US

(73) Assignee: YESBIO CO., LTD., Goyang-si, Gyeonggi-do (KR)

(21) Appl. No.: 12/450,439

(22) PCT Filed: Mar. 17, 2008
(86) PCT No.: PCT/KR2008/001500

§ 371 Date: Sep. 25, 2009

(30) Foreign Application Priority Data
Mar. 26, 2007 (KR) 10-2007-0029058
Mar. 14, 2008 (KR) 10-2008-0023982

(51) Int. Cl. A61F 2/28; A61C 8/00; A61K 9/70

(52) U.S. Cl. 424/443; 433/215; 623/23.61

(57) ABSTRACT

A barrier membrane for guided bone regeneration and a method of manufacturing the same are provided. The barrier membrane for guided bone regeneration that is made of silver, gold, or gold alloy includes a substrate having texture including protrusions with a predetermined shape, a polymer layer formed by coating an upper surface of the substrate with polymer solution, and a bio-ceramic layer formed by coating a lower surface of the substrate. It is possible for the barrier membrane for guided bone regeneration to secure biocompatibility, exclusion and sealing of cells, space maintenance, connectivity with tissues, and easiness of using the barrier membrane which are required for guided bone/tissue regeneration (GBR/GTR).

### TECHNICAL FIELD

The present invention relates to a barrier membrane for guided bone and tissue regeneration, and more particularly, a barrier membrane for guided bone and tissue regeneration capable of securing biocompatibility, exclusion and sealing of cells, space maintenance, connectivity with tissues, and easiness of using the barrier membrane by forming a polymer layer and a bio-ceramic layer respectively on upper and lower surfaces of a substrate made of gold (Au) which has texture including protrusions with predetermined shapes.

### BACKGROUND ART

In order to regenerate tissues destroyed and disappeared due to periodontal diseases, a surgical operation or non-surgical operation may be performed. Bone graft and guided tissue regeneration (GTR) for regenerating the destroyed periodontal tissues are clinically performed as the surgical operation. However, in a case where bones are damaged due to the periodontal diseases, an alveolar bone is not recovered by attaching the epithelium through a simple periodontal curettage or operation. Accordingly, the GTR for guiding formation of bone by using a barrier membrane for periodontal tissue regeneration has been widely employed.

The GTR that has been clinically used since 1986 is referred to regeneration of a new periodontal ligament formed by cells originated from a periodontal ligament which is attached to new cementum. In the GTR, gingival epitheliums and connective tissues are excluded by inserting a barrier membrane between a root surface and a valve which are cleansed when performing flap surgery, and it is possible to attach new periodontal tissues by moving only periodontal ligament cells and periodontal bone cells under a wounded part to the wounded part. The barrier membrane is used for the aforementioned regeneration. The barrier membrane is used to prevent epithelium cells from moving to a root apex, prevents gingival fibers from penetrating into a damaged part, and induces growth of cells from periodontal ligaments and a periodontal bone.

The barrier membrane used for the GTR may be divided into absorbent and unabsorbent barrier membranes.

Since a structure and strength of the unabsorbent barrier membrane is maintained when the unabsorbent barrier membrane exists in tissues, it is possible to completely control the unabsorbent barrier membrane after the GTR. The unabsorbent barrier membrane secures a relatively constant clinical effect. However, the unabsorbent barrier membrane has to be removed after the GTR. When the unabsorbent barrier membrane is not removed, an inflammation of new tissues may be caused by the unabsorbent barrier membrane. Expanded polytetrafluoroethylene (e-PTFE) that is a material of a Gore-tex regenerative membrane of Gore medical corporation in the USA may be used for the unabsorbent barrier membrane. However, since the Gore-tex regenerative membrane is very expensive, the Gore-tex regenerative membrane has not been widely used.

On the other hand, the absorbent barrier membrane accommodates patients without an additional operation for removing the absorbent barrier membrane, thereby reducing costs. Accordingly, recently, research on the absorbent barrier membrane has been actively performed. However, reaction of tissues may be unavoidably caused due to resolvability of the absorbent barrier membrane in a human body. In case of a material with high absorptivity, an inflammation reaction may occur. In addition, it is advantageous that the absorbent barrier membrane is resolved in the human body. The absorbent barrier membrane is deficient in strength and shape allowance. Thus, the absorbent barrier membrane dose not satisfy a condition that a structure with a predetermined shape (in vivo structure) has to be maintained in the human body for 4 to 6 weeks based on a biological principle.

Accordingly, a barrier membrane that secures biocompatibility, exclusion and sealing of cells, space maintenance, connectivity with tissues, and easiness of using the barrier membrane is requested to be developed.

### DISCLOSURE

### Technical Problem

The present invention provides a barrier membrane for guided bone regeneration that is an unabsorbent barrier layer that secures biocompatibility, exclusion and sealing of cells, space maintenance, connectivity with tissues, and easiness of using the barrier membrane

### Technical Solution

According to a first aspect of the present invention, there is provided a barrier membrane for guided bone regeneration comprising a substrate having a previously set thickness, wherein the substrate is made of gold, silver, or gold alloy obtained by adding an element such as platinum (Pt), palladium (Pd), silver (Ag), copper (Cu), iridium (Ir), zinc (Zn), indium (In), rhodium (Rh), tin (Sn), and the like to gold, and wherein the substrate has texture with arbitrary shapes.

According to a second aspect of the present invention, there is provided a barrier membrane for guided bone regeneration comprising a substrate having a previously set thickness, wherein the substrate is made of gold, silver, or gold alloy obtained by adding an element such as platinum (Pt), palladium (Pd), silver (Ag), copper (Cu), iridium (Ir), zinc (Zn), indium (In), rhodium (Rh), tin (Sn), and the like to gold.

In the above aspects of the present invention, the barrier membrane for guided bone regeneration may further include a polymer layer containing an antibiotic on an upper surface of the substrate. In addition, the barrier membrane for guided bone regeneration may further include a bio-ceramic layer obtained by coating a lower surface of the substrate with bio-ceramic. In addition, the bio-ceramic layer may be formed by coating the lower surface of the substrate with a material selected from the group consisting of hydroxyapatite (HA), β-tricalcium phosphate (β-TCP), bioglass, and gypsum by spraying the material onto the lower surface in high pressure. In addition, the bio-ceramic layer may be made of a material selected from the group consisting of hydroxyapatite (HA), β-tricalcium phosphate (β-TCP), bioglass, and gypsum, and the bio-ceramic layer may be attached to the lower surface of the substrate by using a polymer material having adhesive force with the selected material.

In addition, the texture of the barrier membrane for guided bone regeneration according to the first aspect may include a plurality of protrusions and protruded pores, and the protruded pores may pass through the upper and lower surfaces of the substrate. In addition, the substrate of barrier membrane for guided bone regeneration according to the above aspects may have the previously set thickness and strength by being rolled.

### ADVANTAGEOUS EFFECTS

It is possible to provide a barrier membrane having high biocompatibility and space maintenance by making the barrier membrane for guided bone regeneration with gold or silver. In addition, the barrier membrane for guided bone regeneration is constructed with a substrate made of silver, gold, or gold alloy. It is possible to secure biocompatibility, exclusion and sealing of cells, space maintenance, connectivity with tissues, and easiness of using the barrier membrane by forming a polymer layer and a bio-ceramic layer respectively on upper and lower surfaces of the substrate. In addition, it is possible to prevent a flow of soft tissues in a junction part with the soft tissues and minimize bursts of sutures due to shrinkage of tissues by forming texture including protrusions on the substrate according to an embodiment of the present invention. In addition, it is possible for blood to circulate by forming protruded pores on the substrate, thereby supplying blood to epithelium cells.

Furthermore, the barrier membrane for guided tissue regeneration according to an embodiment of the present invention may be used to induce regeneration of bone tissues for orthopedic or plastic surgery treatments, in addition to dental treatments. Accordingly, the barrier membrane for guided tissue regeneration may be used for various purposes.

### DESCRIPTION OF DRAWINGS

FIG. 1 is a schematic diagram illustrating a structure of a barrier membrane for guided tissue regeneration according to a first embodiment of the present invention.

FIG. 2 is a schematic diagram illustrating a structure of a barrier membrane for guided tissue regeneration according to a second embodiment of the present invention.

FIG. 3 is a top plan view illustrating the barrier membrane for guided bone regeneration having protrusions and protruded pores according to the second embodiment of the present invention.

FIG. 4 includes cross sectional views taken along the directions A-A′ and B-B′ of the barrier membrane for guided tissue regeneration of FIG. 3.

FIG. 5 is a flowchart of an example of a procedure of manufacturing the barrier membrane for guided bone regeneration according to the second embodiment of the present invention.

### BEST MODEL

### First Embodiment

Hereinafter, a structure and an operation of a barrier membrane for guided bone and tissue regeneration according to a first embodiment of the present invention will be described with reference to the attached drawings.

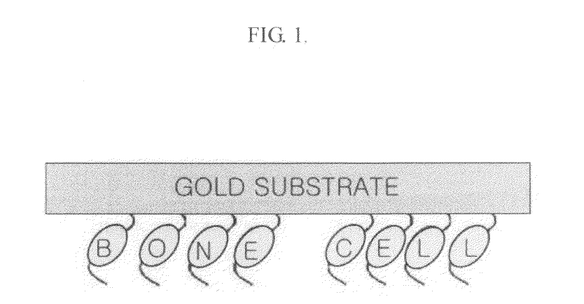

FIG. 1 is a schematic diagram illustrating a structure of a barrier membrane for guided bone and tissue regeneration according to a first embodiment of the present invention. Referring to FIG. 1, a barrier membrane 10 for guided bone and tissue regeneration according to the first embodiment is constructed with a substrate made of gold or silver having high purity so as to improve biocompatibility and space maintenance or constructed with a substrate made of gold alloy obtained by adding an element such as platinum (Pt), palladium (Pd), silver (Ag), copper (Cu), iridium (Ir), zinc (Zn), indium (In), rhodium (Rh), tin (Sn), and the like to gold so as to improve strength and functions. Specifically, it is possible to provide a barrier membrane with high space maintenance in addition to high biocompatibility by using a substrate made of a gold material for the barrier membrane.

In addition, the substrate may have a previously set thickness through a rolling procedure. At this time, the previously set thickness may range from 0.015 to 0.15 mm. The thickness of the substrate has to be equal to or greater than 0.015 mm so as to secure space maintenance of the barrier membrane. In addition, in order to remove foreign body sensation due to the barrier membrane, the thickness of the substrate may be equal to or less than 0.15 mm. If the thickness of the substrate of the barrier membrane is greater than 0.15 mm, it is possible to secure space maintenance. However, it is not easy to insert the barrier membrane. The barrier membrane may cause damage of soft tissues. In addition, a tongue may feel a foreign body under the soft tissues.

On the other hand, the barrier membrane according to the first embodiment of the present invention effectively induces regeneration of tissues of periodontal bone by further including a bio-ceramic layer under the substrate. The lower surface of the substrate is coated with the bio-ceramic layer by spraying bio-ceramic onto the lower surface of the substrate in high pressure. The bio-ceramic layer used for the barrier membrane according to the current embodiment may be made of a bio-ceramic material having a function of guiding regeneration of periodontal bone tissues, such as hydroxyapatite (HA), β-tricalcium phosphate (β-TCP), bioglass, gypsum, and the like.

In addition, the barrier membrane according to the first embodiment of the present invention may further include a polymer layer on the upper surface of the substrate. The polymer layer is formed by coating the upper surface of the substrate with bio-polymer solution containing an antibiotic. The antibiotic contained in the polymer layer suppresses an inflammation and a secondary infection which may occur after a surgical operation. The antibiotic contained in the polymer layer of the barrier layer according to an embodiment of the present invention may be tetracycline.

### Second Embodiment

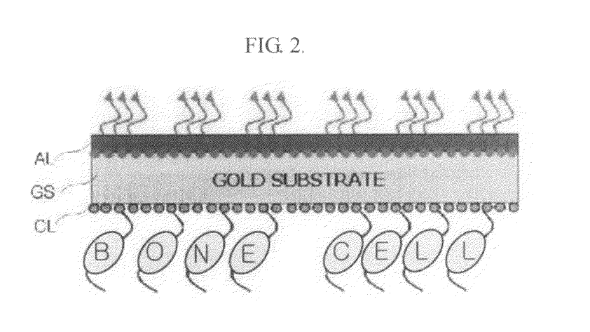

Hereinafter, a structure and an operation of a barrier membrane for guided bone and tissue regeneration according to a second embodiment of the present invention will be described with reference to FIGS. 2 to 4.

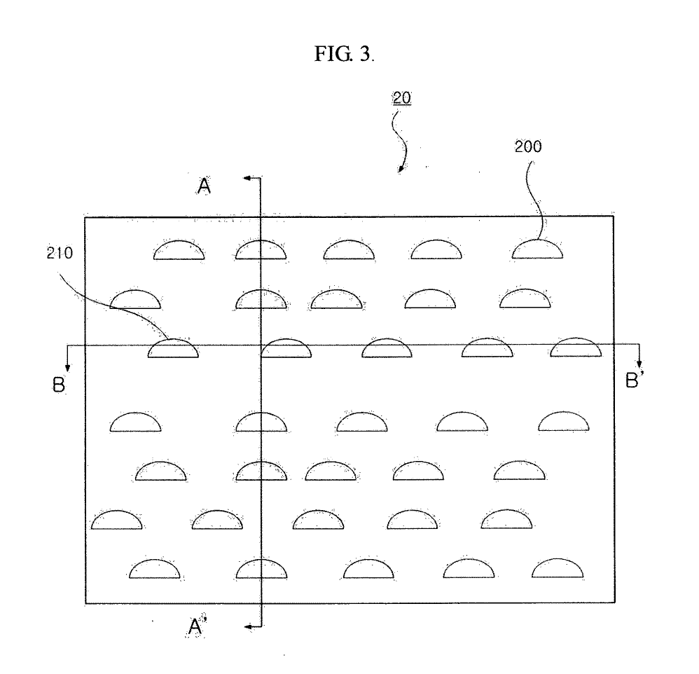

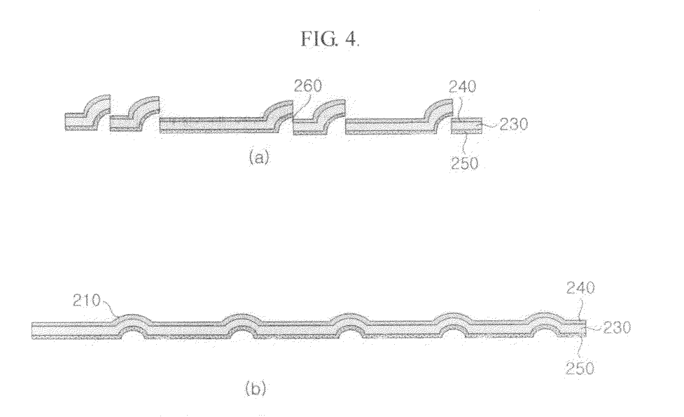

FIG. 2 is a schematic diagram illustrating a structure of a barrier membrane for guided bone and tissue regeneration according to a second embodiment of the present invention. FIGS. 3 and 4 include a top plan view and cross sectional views illustrating the barrier membrane according to the second embodiment of the present invention.

Referring to FIGS. 3 and 4, the barrier membrane 20 for guided bone regeneration according to the first embodiment of the present invention includes a substrate 230, a polymer layer 240 containing an antibiotic, and a bio-ceramic layer 250.

The substrate 230 is made of gold or silver having high purity so as to improve biocompatibility and space maintenance or made of gold alloy obtained by adding an element such as platinum (Pt), palladium (Pd), silver (Ag), copper (Cu), iridium (Ir), zinc (Zn), indium (In), rhodium (Rh), tin (Sn), and the like to gold so as to improve strength and functions.

In FIG. 4, (a) is a cross sectional view taken along the direction A-A′ illustrating the barrier membrane of FIG. 3, and (b) is a cross sectional view taken along the direction B-B′ illustrating the barrier membrane of FIG. 3. Referring to FIG. 4, the substrate 230 has texture including a plurality of protrusions 200 and 210 and protruded pores 260. The barrier membrane for guided bone and tissue regeneration according to the second embodiment prevents a flow of soft tissues in a junction part with the soft tissues by having the texture including hemispheric protrusions. In addition, the barrier membrane according to the second embodiment enables blood to be supplied to epithelial cells by forming the protruded pores in the texture of the substrate.

On the other hand, although the texture including a plurality of hemispheric protrusions is exemplified in the second embodiment, it will be understood by those skilled in the art that a plurality of cylindrical protrusions, a plurality of cone type protrusions, or a plurality of polygonal solid protrusions may be employed for the texture. In addition, in the barrier membrane according to the second embodiment, the substrate may have various types of texture in addition to the texture including the protrusions and the protruded pores. For example, the substrate may have texture including a plurality of through holes or texture including only protrusions.

The lower surface of the substrate is coated with the bio-ceramic layer 250 by spraying bio-ceramic onto the lower surface of the substrate in high pressure so as to guide regeneration of periodontal bone tissue. The bio-ceramic layer used for the barrier membrane according to the second embodiment may be made of a bio-ceramic material having a function of guiding regeneration of periodontal bone tissue, such as hydroxyapatite (HA), β-tricalcium phosphate (β-TCP), bioglass, gypsum, and the like.

The polymer layer 240 is formed by coating the upper surface of the substrate with bio-polymer solution containing an antibiotic. The antibiotic contained in the polymer layer suppresses an inflammation and a secondary infection which may occur after a surgical operation. The antibiotic contained in the polymer layer of the barrier layer according to an embodiment of the present invention may be tetracycline.

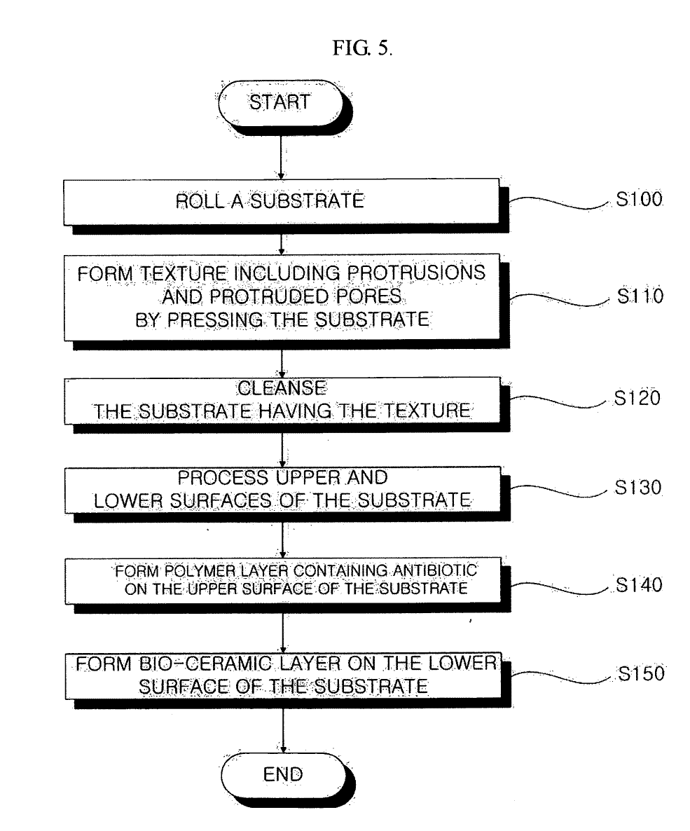

Hereinafter, an example of a procedure of manufacturing the barrier membrane 20 according to the second embodiment of the present invention will be described with reference to FIG. 5.

First, strength of the substrate 230 is increased by rolling the substrate 230 made of silver or gold having high purity or gold alloy containing an element such as platinum (Pt), palladium (Pd), silver (Ag), copper (Cu), iridium (Ir), zinc (Zn), indium (In), rhodium (Rh), tin (Sn), and the like by using a roller to a thickness ranging from 0.015 to 0.15 mm (operation 100).

Next, the substrate 230 has texture including hemispheric protrusions 210 and 200 and protruded pores 260 by pressing the substrate 230 (operation 110). Here, the texture is used to prevent a flow of soft tissues such as gingival.

Hereinafter, the rolled substrate is cleansed by using an ultrasonic cleaner (operation 120).

The upper and lower surfaces of the rolled substrate 230 are sandblasted so as to be roughly processed (operation 130).

When the sandblasting process is completed, the upper surface of the substrate having the texture 204 is spray-coated with polymer solution containing an antibiotic so as to form the polymer layer 240 containing the antibiotic on the upper surface of the substrate (operation 140). Here, the polymer solution containing the antibiotic is produced by mixing a solvent with polymers to produce polymer solution and mixing the polymer solution with an antibiotic such as tetracycline.

As described above, when the polymer layer 240 containing the antibiotic is formed on the upper surface, the bio-ceramic layer 250 is formed on the lower surface of the substrate 230 by spraying a bio-ceramic such as hydroxyapatite (HA), β-tricalcium phosphate (β-TCP), bioglass, gypsum, and the like onto the lower surface of the substrate in high pressure and plasma-coating the lower surface of the substrate with the bio-ceramic (operation 150).

In addition, the bio-ceramic may be attached by using an adhesive polymer material. Any bio-polymer material may be used as the polymer material of for this case. Preferably, one of polylactide-co-glycolide (PLGA), poly-L-lactic acid (PLLA), polyglycolide (PGA), Cyanoacrylate may be employed as the polymer material.

As described above, manufacturing of the barrier membrane for guided tissue regeneration according to the second embodiment is completed by forming the bio-ceramic layer 250 on the lower surface of the substrate.

While the present invention has been particularly shown and described with reference to exemplary embodiments thereof, it will be understood by those skilled in the art that various changes in form and details may be made therein without departing from the spirit and scope of the present invention as defined by the appended claims.

### INDUSTRIAL APPLICABILITY

The barrier membrane for guided bone and tissue regeneration according to an embodiment of the present invention may be used to induce regeneration of bone tissues in orthopedic or plastic surgery treatments, in addition to dental treatments. Accordingly, the barrier membrane for guided bone and tissue regeneration may be used for various purposes.

1. A barrier membrane for guided bone and tissue regeneration comprising a substrate having a previously set thickness,
wherein the substrate is made of gold, silver, or gold alloy obtained by adding an one element of platinum (Pt), palladium (Pd), silver (Ag), copper (Cu), iridium (Ir), zinc (Zn), indium (In), rhodium (Rh) and tin (Sn) to gold, and
wherein the substrate has texture with arbitrary shapes.

2. A barrier membrane for guided bone and tissue regeneration comprising a substrate having a previously set thickness, wherein the substrate is made of gold, silver, or gold alloy obtained by adding an one element of platinum (Pt), palladium (Pd), silver (Ag), copper (Cu), iridium (Ir), zinc (Zn), indium (In), rhodium (Rh) and tin (Sn) to gold.

3. The barrier membrane for guided bone and tissue regeneration according to claim 1, further comprising a polymer layer containing an antibiotic on an upper surface of the substrate.

4. The barrier membrane for guided bone and tissue regeneration according to claim 1, further comprising a bio-ceramic layer obtained by coating a lower surface of the substrate with bio-ceramic.

5. The barrier membrane for guided bone and tissue regeneration according to claim 1,
wherein the texture includes a plurality of protrusions and protruded pores, and
wherein the protruded pores pass through the upper and lower surfaces of the substrate.

6. The barrier membrane for guided bone and tissue regeneration according to claim 4, wherein the bio-ceramic layer is formed by coating the lower surface of the substrate with a material selected from the group consisting of hydroxyapatite (HA), β-tricalcium phosphate (β-TCP), bioglass, and gypsum by spraying the material onto the lower surface in high pressure.

7. The barrier membrane for guided bone and tissue regeneration according to claim 4,
wherein the bio-ceramic layer is made of a material selected from the group consisting of hydroxyapatite (HA), β-tricalcium phosphate (β-TCP), bioglass, and gypsum, and
wherein the bio-ceramic layer is attached to the lower surface of the substrate by using a polymer material having adhesive force with the selected material.

8. The barrier membrane for guided bone regeneration according to claim 1, wherein the substrate is rolled so as to have the previously set thickness.

9. The barrier membrane for guided bone and tissue regeneration according to claim 2, further comprising a polymer layer containing an antibiotic on an upper surface of the substrate.

10. The barrier membrane for guided bone and tissue regeneration according to claim 2, further comprising a bio-ceramic layer obtained by coating a lower surface of the substrate with bio-ceramic.

11. The barrier membrane for guided bone and tissue regeneration according to claim 10, wherein the bio-ceramic layer is formed by coating the lower surface of the substrate with a material selected from the group consisting of hydroxyapatite (HA), β-tricalcium phosphate (β-TCP), bioglass, and gypsum by spraying the material onto the lower surface in high pressure.

12. The barrier membrane for guided bone and tissue regeneration according to claim 10
wherein the bio-ceramic layer is made of a material selected from the group consisting of hydroxyapatite (HA), β-tricalcium phosphate (β-TCP), bioglass, and gypsum, and
wherein the bio-ceramic layer is attached to the lower surface of the substrate by using a polymer material having adhesive force with the selected material.

13. The barrier membrane for guided bone regeneration according to claim 2, wherein the substrate is rolled so as to have the previously set thickness.
